(19) United States
(12) Patent Application Publication
(10) Pub. No.: US 2002/0127256 A1
(43) Pub. Date: Sep. 12, 2002

(54) Compositions and methods for treating dermatological disorders

(76) Inventor: Howard Murad, Marina del Rey, CA (US)

Correspondence Address: PENNIE & EDMONDS LLP, 1667 K STREET NW, SUITE 1000, WASHINGTON, DC, 20006

(21) Appl. No.: 10/080,717

(22) Filed: Feb. 25, 2002

Related U.S. Application Data
(60) Provisional application No. 60/272,046, filed on Mar. 1, 2001.

(51) Int. Cl. A61K 35/78; A61K 31/7024; A61K 31/192; A61K 31/198; A61K 7/00

(52) U.S. Cl. 424/401; 424/768; 424/776; 514/23; 514/54; 514/557; 514/570; 514/561; 514/21

(57) ABSTRACT

The invention relates to dermatological agents and methods for treating dermatological disorders. The dermatological agents include a therapeutically effective amount of at least one acid selected from ellagic acid, ferrulic acid, caffeic acid, or tannic acid in an amount sufficient to strengthen cell membranes in the skin. The at least one acid is preferably combined with at least one of a moisturizing agent, an anti-inflammatory component, an immunity boosting component or an additional anti-oxidant, and a pharmaceutically acceptable carrier.

### TECHNICAL FIELD

The invention relates to dermatological agents containing one or more of ellagic acid, tannic acid, ferullic acid, or caffeic acid and methods of using the same to treat dermatological disorders.

### BACKGROUND OF THE INVENTION

Human skin is a composite material of the epidermis and the dermis. The topmost part of the epidermis is the stratum corneum. This layer is the stiffest layer of the skin, as well as the one most affected by the surrounding environment. Below the stratum corneum is the internal portion of the epidermis. Below the epidermis, the topmost layer of the dermis is the papillary dermis, which is made of relatively loose connective tissues that define the micro-relief of the skin. The reticular dermis, disposed beneath the papillary dermis, is tight, connective tissue that is spatially organized. The reticular dermis is also associated with coarse wrinkles. At the bottom of the dermis lies the subcutaneous layer.

The principal functions of the skin include protection, excretion, secretion, absorption, thermoregulation, pigmentogenesis, accumulation, sensory perception, and regulation of immunological processes. These functions are detrimentally affected by the structural changes in the skin due to aging and excessive sun exposure. The physiological changes associated with skin aging include impairment of the barrier function and decreased turnover of epidermal cells, for example. [Cerimele, D., et al., Br. J. Dermatol, 122 Suppl. 35, p. 13-20 (April 1990)].

The mechanical properties of the skin, such as elasticity, are controlled by the density and geometry of the network of collagen and elastic fiber tissue therein. Damaged collagen and elastin lose their contractile properties, resulting in skin wrinkling and skin surface roughness. As the skin ages or becomes unhealthy, it acquires sags, stretch marks, bumps, bruises or wrinkles, it roughens, and it has reduced ability to synthesize Vitamin D. Aged skin also becomes thinner and has a flattened dermoepidermal interface because of the alterations in collagen, elastin, and glycosaminoglycans. [Fenske, N. A, and Lober, C. W., J. Am. Acad. Dermatol., 15:571-585 (October 1986); Montagna, W. and Carlisle, K., Journal of Investigative Dermatol., 73(1):47-53 (1979)].

The skin is the most environmentally-stressed organ in mammals, particularly in humans. The skin is exposed to a wide variety of biological, chemical, and physical attacks including, for example, exposure to toxic chemicals and hostile environments and is also the only organ directly exposed to ultraviolet (“UV”) light in the presence of oxygen. [See, e.g., P. Mayer, et al., Cosmetic & Toiletries, 108:99-109 (February 1993)]. Lengthy exposure of the skin to UV light typically damages the skin, resulting in sunburn, photoaging, and carcinogenesis.

UV light exposure in the presence of oxygen results in the creation of free radicals. In the skin, these radicals frequently trigger the release of inflammatory mediators, commonly manifested as sun bum; cytoskeletal alterations, breaking down the collagen in the skin; and may also result in structural DNA changes, such as DNA strand breaks and dimer formation. [K. Werninghaus, et al., Arch Dermatol., 130:1257-1261 (October 1994)]. The body attempts to neutralize the free radicals generated by UV light through the use of antioxidants. Antioxidants are commonly found in two forms: enzymatic and non-enzymatic. Superoxide dismutase (SOD), catalase, and glutathione peroxidase are some of the natural enzymatic antioxidants used by the body. SOD accelerates the spontaneous reduction of superoxide free radicals into peroxides and oxygen. Catalase then further decomposes hydrogen peroxide into water and oxygen. Finally, the glutathione peroxidase reduces both hydrogen peroxide and free organic hydroperoxides. Some non-enzymatic antioxidants, such as Vitamin E (tocopherol), Vitamin A (beta-carotene), and Vitamin C (ascorbic acid) have each been individually applied to assist the skin in scavenging free radicals and neutralizing the harmful effects of UV light. [P. Pugliese, “A Brief Introduction to Free Radicals and Oxygen Stress,” Paper presented at International Conference of Aesthetics and Dermatology, Los Angeles, (February 1991)]. Conventional skin protection efforts typically attempt to either shield the skin from UV light to prevent the production of free radicals or provide additional agents capable of neutralizing the free radicals.

Topical applications are one such effort known in the art to help shield the skin from the sun's harmful UV effects. These sun-screens often are water- or oil-based lotions or ointments that incorporate photo-protectant materials such as titanium and zinc oxide. [J. Weiss, Skin, 16-23 (March/April 1996)]. Although the most widely used form of protection against exposure to sunlight, these topical applications tend to suffer from several drawbacks. First, large amounts of photo-protective materials are usually incorporated into the topical applications, some of which have recently become suspected of having toxicity or otherwise being harmful under these conditions. Furthermore, the effectiveness of such topical applications is dependent upon a constant and uniform coverage of the skin, which is often difficult to obtain. Many individuals fail to use these topical sunscreens on a regular or continuing basis, as is required under prolonged UV exposure. Moreover, sunscreens and other topical applications do not consistently provide good protection for all types of UV light. [Id.]. Finally, sun screens are ineffective at treating skin already damaged by exposure to UV light.

A variety of vitamins and minerals have in individually been administered to treat certain skin and other problems that occur when the patient has a deficiency of that vitamin or mineral. Vitamin A, for example, assists in the treatment of acne and to facilitate wound healing; vitamin C (ascorbic acid) assists in the prevention of skin bruising and wound healing; vitamin E is an antioxidant; and copper assists in the treatment of elastic tissue defects. [Neldner, K. H., Amer. Acad. Derm. Annl. Mtg., Wash D.C., Dec. 6, 1993]. Topical use of vitamin C is also believed to ward off sun damage, reduce breakdown of connective tissues, and possibly promote collagen synthesis. [Thomas, P., Medical World News, p. 12, March 1991]. Vitamin E is used topically as an anti-inflammatory agent, for enhancement of skin moisturization, for UV-ray protection of cells, and for retardation of premature skin aging.

It is thought that minerals are typically needed to maintain the effectiveness of the body's enzymatic antioxidants. Both copper and zinc are thought to be necessary in the proper functioning of SOD. [G. La Ruche & J.-P. Cesarini, Photodermatol Photoimmunol Photomed., 8:232-235 (1991)]. Manganese is believed to be a cofactor in the mitochondrial form of SOD. Also, selenium is thought to be necessary for glutathione peroxidase activity, one of the enzymatic antioxidants found naturally in the body. Unfortunately, few experiments into the skin-protecting effects of these antioxidants have provided scientific or conclusive results.

In particular, a study that orally administered vitamin E supplements to participants and then tested their response to the sun found that Vitamin E did not mitigate the UV damage, despite the fact that the subjects were given thirteen times the recommended daily allowance. [K. Weminghaus, et al., Arch. Dermatol., 130:1257-1261 (October 1994)]. Furthermore, beta-carotene has been reported to have beneficial effects in some studies, but has had no effect in others. Finally, another study noted the photo-protective effect of the oral administration of butylated hydroxy toluene, but little effect was shown using vitamins C or E.

Certain herbs have also been found helpful in protecting the skin from the sun's harmful effects. Herb extracts such as burdock root, echinacea, yellow dock root and grape seeds posses detoxifying properties that have been individually applied to help the body eliminate harmful free radicals. Burdock root contains the active ingredient inulin, and is useful in treating cancerous skin conditions, as well as inflammation. Echinacoside and caffeoyl derivatives present in echinacea act as antioxidants, which protect the skin when applied topically. [R. Facino, et al., Planta Med. 61:510-514 (1995)]. Yellow dock root contains the active constituent chrysarobin, which has been used in the treatment of chronic skin diseases, such as eczema, leprosy, psoriasis, and cancer. [M. Tierra, “Planetary Herbology,” p. 194 (1988)]. Potent bioflavanoids, known as oligomeric proanthanols and proanthocyanidins (OPC's), are found in flowers, plant leaves, and grape seeds. [Lubell, A., Cosmetic Dermatol., 9(7):58& 60 (July 1996)]. These OPC's are thought to be potent antioxidants possessing 20 times the antioxidant power of vitamin C and 50 times the antioxidant power of vitamin E. These herbs have been individually used both topically and orally to protect the skin from various afflictions.

Other studies have attempted to demonstrate the synergistic effect of a mixture of antioxidants. In one study, the subjects were given selenium and copper along with a vitamin supplement of vitamin A and E. [G. La Ruche & J. P. Cesarini, Photodermatol Photoimmunol Photomed., 8:232-235 (1991)]. Although the supplements did protect the skin cells to some extent against ultraviolet-induced cell damage, they did not prevent light-induced erythema, i.e., redness.

N-Acetylglucosamine and glucosamine have been examined for use in the prevention and treatment of degenerative joint diseases and cartilage loss, and found to increase the glycosaminoglycans present in the cartilage to restore cartilage. [See Grevenstein, J., et al., Acta Orthopaedia Belgica, 57(2):157-161 (1991); Setnikar, I., Drug Res., 36(4):720-733 (1986); Drovanti, A., et al, Clin. Therap., 3(4):1-6 (1980)]. Glucosamine has also been examined in connection with arthritis and oral and injected glucosamine have been reported to be useful for arthrosic patients. [Tapadinhas, M. J., et al., Pharmatherapeutica, 3(3):157-168 (1982); D'Ambrosio, E., et al., Pharmatherapeutica, 2(8):504-508 (1981)].

The metabolism of glycosaminoglycans under the influence of herbal and other anti-inflammatory agents has been examined by measuring glycosaminoglycans in the skin, liver, kidney, and spleen after administration of several compounds. [Reddy, G. K., et al., Biochem. Pharmacology, 38(20):3527-3534 (1989)].

In addition to their individual use to supplement a deficiency in a patient, various of the above ingredients have been combined to form pharmaceuticals designed to prevent and treat certain cellular, skin, and other conditions. For example, U.S. Pat. No. 3,773,930 discloses a low residue, dietary composition having at least one amino acid and a quantity of non-amino acid derived caloric material sufficient to obviate the diarrhea problem of straight amino acid compositions. A flavoring material may also be included to render the composition more palatable.

U.S. Pat. No. 4,285,964 discloses a salt of (+)-catechin formed by reacting (+)-catechin with at least a basic amino acid, such as L-lysine and L-arginine; and a hydrosoluble double salt formed from the reaction product of (+)-catechin with a basic amino-acid, such as L-lysine and L-arginine, and another inorganic or organic acid. The patent further discloses methods of treating degenerative diseases of the connective tissue by topically administering the composition.

U.S. Pat. No. 4,414,202 discloses a composition for the treatment of skin wounds with a buffered salt solution having a pH between 6 to 7.8 and administering a starch hydrolysate compound, and preferably including alphaketoglutaric acid or alphaketoglutarate salts. Optional additives to the composition include ascorbic acid or salts thereof, ferrous salts, and glycine, L-Proline, and L-Lysine.

U.S. Pat. No. 4,424,232 discloses a topical composition for the treatment of herpes simplex, cold sores, lesions, and other painful skin conditions including L-lysine, gibberellic acid, and urea in an inert carrier having water. The composition may also include L-ascorbic acid, as well as methyl paraben, propyl paraben, or mixtures thereof.

U.S. Pat. No. 4,647,453 discloses a method and composition for treatment of tissue degenerative inflammatory disease in animals and humans by oral administration of ascorbic acid, bioavailable calcium, a precursor or stimulant of epinephrine or norepinephrine of tyrosine or phenylalanine, and an anti-inflammatory substance selected from anti-inflammatory sugars, amino sugars and biocompatible acid addition salts thereof, and anti-inflammatory amino acids, to promote connective tissue regrowth.

U.S. Pat. No. 5,141,741 to Ishida et al. discloses an anti-sunburn skin care composition containing a polyvalent metal salt of an ellagic acid compound and a cosmetic carrier, as well as a method of protecting human skin from sunburn by applying the composition to the human skin.

U.S. Pat. No. 5,198,465 discloses a composition for treating precursor deficiencies in the synthesis of collagen with proline, glycine, lysine, vitamin C, and one or more compounds selected from a-ketoglutaric acid, methionine, cysteine, cystine, valine, and pharmaceutically acceptable diluents and excipients.

U.S. Pat. Nos. 5,332,579 and 5,308,627 disclose a nutritional supplement to assist persons recovering from addiction by administering a variety of vitamins and minerals including enzyme activating substances such as magnesium and zinc; an enzyme co-factor that is a vitamin like various vitamin B complexes; an enzyme producer such as an amino acid like glutamic acid; an herbal antispasmodic substance like Valerian root; and vitamin C.

U.S. Pat. No. 5,415,875 discloses a method of suppressing formation of lipid peroxide and removing peroxide by applying to the skin a decomposed product of shell membrane and tocopherol and derivatives. Lysine, proline, Vitamin C, for examples, are listed among a vast genus of optional additives.

U.S. Pat. No. 5,994,403 to Donatiello discloses tannic acid solutions as a treatment for fungal infections of the skin.

The Journal of the Science of Food and Agriculture, 79 476-480 (1999), Saija, et al. disclose the antioxidant activity of caffeic and ferulic acids as a means of mediating photooxidative stress in skin. Caffeic and ferulic acids were reported to be suitable candidates as topical protective agents against UV radiation-induced skin damage.

The International Journal of Cancer, 51, 425-432, (1992), Gali et al. disclose the antioxidant and antitumor promotion activities in skin of several hydrolyzable tannins, including tannic acid and ellagic acid. The antioxidant properties of such tannins were determined to be necessary but not sufficient for anti-tumor-promotion activity.

The International Journal of Pharmaceutics, 199, 39-47 (2000), Saija, et al. disclose the topical application of antioxidants such as caffeic and ferulic acids for protecting skin against UV-mediated oxidative damage.

The Biochemcial and Biophysical Research Communications, 114 (1), 388-394 (1983), Del Tito, et al. disclose that ellagic acid is a potent inhibitor of epidermal microsomal aryl hydrocarbon hydroxylase and inhibits carcinogen metabolism in skin. The reference reports that further study is needed is needed to determine if phenolic compounds such as ellagic acid may prove useful in modulating the risk of cutaneous cancer from environmental chemicals.

Plant Polyphenols, Plenum Press, Hemingway and Laks, Eds., 783-801, (1992), Perchellet et al. disclose the antimutagenic and anticarcinogenic activity of the naturally occurring plant phenols, tannic acid and ellagic acid. These phenols were reported to inhibit known biochemical markers associated with tumor promotion. Tannic acid and other polyphenols are disclosed as being potentially valuable in cancer therapy and/or prevention.

Nutrition and Cancer, 32(2), 81-85, (1998), A. Kaul et al. discloses that ellagic acid, tannic acid, caffeic acid, and ferulic acid may be effective anticarcinogens since topical application of these acids with phorbol-12-myristate-13-acetate or mezerein reportedly results in significant protection against 7,12-dimethyl-benza[a]anthracene induced skin tumors in mice.

Biomaterials, 18, 749-754, (1997), F. H. Heijmen et al. discloses that collagen can be cross-linked with tannic acid and that the cross-linking is influenced by the presence of metal ions.

International Journal of Cosmetic Science, 22, 291-303, (2000), H. Shimogaki et al. discloses that ellagic acid inhibits tyrosinase and suppresses skin pigmentation in guinea pigs and may be used as a skin whitening agent.

The above references, however, do not teach pharmaceutical compositions or methods for improving skin wrinkles along with other conditions, such as skin elasticity and softness. Thus, it is desired to find a pharmaceutical composition and a method for the treatment of wrinkles and other skin conditions associated with aging. The present invention advantageously provides pharmaceutical compositions, as well as methods of treatment comprising the administration of such compositions, to repair aged skin and to treat wrinkles and other skin disorders.

### SUMMARY OF THE INVENTION

The present invention relates to a dermatological agent for treating a dermatological condition in a patient having skin. The dermatological agent includes at least one acid comprising at least one of ferrulic acid, caffeic acid, and tannic acid and present in an amount sufficient to strengthen cell membranes in the skin; at least one moisturizing agent present in an amount sufficient to facilitate hydration of the skin; and a pharmaceutically acceptable carrier.

The at least one acid may be present in an amount from about 0.01 to 80 weight percent of the dermatological agent. The moisturizing agent may be a mono- or poly-hydroxy acid, hydrophobic agent, hydrophilic agent, or mixture thereof. The mono- or poly-hydroxy acid may be glycolic acid, lactic acid, citric acid, or salicylic acid. The hydrophobic agent may be ceramide, borage oil, tocopherol linoleate, dimethicone, or glycerine. The hydrophilic agent may be hyaluronic acid, sodium peroxylinecarbolic acid, wheat protein, or hair keratin amino acids, and mixtures thereof. The moisturizing agent may also be primrose oil, GLA 3, flax seed oil, or a mixtures thereof.

The dermatological agent may further include at least one cysteine component, magnesium component, manganese component, copper component, or selenium component. The optional cysteine component may be N-acetyl cysteine and be present in an amount from about 1 to 10 weight percent. The optional magnesium component may be magnesium ascorbate and be present in an amount from about 1 to 10 weight percent, wherein the magnesium is present in an amount from about 10 to 30 weight percent of the complex The optional manganese component may be manganese ascorbate and be present in an amount from about 0.5 to 10 weight percent, wherein manganese is present in an amount from about 5 to 20 weight percent of the complex. The optional copper component may be copper sebacate and be present in an amount from about 0.01 to 5 weight percent, wherein the copper is present in an amount from about 5 to 20 weight percent of the complex.

The dermatological agent may also include at least one of wild yam root, wild yam extract, yellow dock, bupleurum, poria cocos, gentian root, myrrh gum, hawthorn berry extract, marshmallow root, rosemary extract, black cohosh, soy or ginger. The amount of the optional wild yam root, wild yam extract, marshmallow root, hawthorn berry extract, rosemary extract, or combination thereof may be from about 0.5 to 8 weight percent each. The amount of optional yellow dock may be from about 1 to 30 weight percent. The amount of optional bupleurum, poria cocos, gentian root, myrrh gum, or combination thereof may be from about 1 to 20 weight percent of the composition.

The dermatological agent may also include at least one anti-inflammatory component in an amount sufficient to reduce inflammation of the patient's skin or an antimicrobial agent. The anti-inflammatory component may be present in an amount from about 5 to 40 weight percent and may be at least one of a vitamin E source, a transition metal component, aloe vera gel, aloe vera, licorice extract, pilewort, Canadian willow root, zinc, allantoin, chamomile, hydrocortisone, steroids, non-steroidal anti-inflammatory drugs, or mixtures thereof. The antimicrobial agent may be an antibacterial agent, an antifungal agent, an antihelmintic, or a combination thereof, in an amount sufficient to inhibit, prevent, or kill microbes

The dermatological also include at least one immunity boosting component in an amount sufficient to stimulate the patient's immune system response to prevent or facilitate repair of damaged skin. The immunity boosting component may be present in an amount from about 1 to 20 weight percent and may be echinacea, echinacea extract, golden seal, or a mixtures thereof.

The dermatological agent may also include at least one antioxidant. The antioxidant may be a catechin-based preparation, a vitamin A source, a ginko biloba extract, a silymarin source, a quercetin compound, a vitamin C source, or a carotenoid.

The dermatological agent may be adapted for oral or topical administration.

In another embodiment of the dermatological agent of the invention, the dermatological agent includes at least one acid selected from the group consisting of ellagic acid, ferrulic acid, caffeic acid, and tannic acid in an amount sufficient to strengthen cell membranes in the skin; an anti-inflammatory component in an amount sufficient to inhibit or reduce inflammation; and a pharmaceutically acceptable carrier.

The anti-inflammatory agent may be one or more of aloe vera gel, aloe vera, licorice extract, pilewort, Canadian willow root, zinc, pile wort, arnica, Vitamin E, allantoin, chamomile, hydrocortisone, steroids, or non-steroidal anti-inflammatory drugs. The anti-inflammatory component may be present in an amount from about 0.1 to 2 weight percent of the composition. The dermatological agent may also include one or more transition metal components, such as zinc or an antimicrobial agent. The antimicrobial agent may be an antibacterial agent, an antifungal agent, an antihelmintic, or a combination thereof, in an amount sufficient to inhibit, prevent, or kill microbes. The at least one acid may be caffeic acid, tannic acid, or ellagic acid.

The invention further relates to a method for treating one or more dermatological conditions in a patient having skin. The method involves administering to the patient a therapeutically effective amount of a dermatological agent comprising at least one of ellagic acid, ferrulic acid, caffeic acid, or tannic acid in an amount sufficient to strengthen cell membranes in the skin; and a pharmaceutically acceptable carrier. The dermatological agent may be administered orally or topically and the effective amount may be from about 1 mg to 2,000 mg per day.

The method may further involve administering at least one additional pharmaceutical composition to facilitate treatment of the dermatological condition. The at least one additional pharmaceutical composition may be a moisturizing agent provided in an amount sufficient to facilitate hydration of the skin. The moisturizing agent may be a mono- or poly-hydroxy acid, a hydrophobic agent, hydrophilic agent, primrose oil, GLA 3, flax seed oil, or a mixtures thereof. The mono- or poly-hydroxy acid may be glycolic acid, lactic acid, citric acid, salicylic acid, or a mixtures thereof. The hydrophobic agent may be ceramide, borage oil, tocopherol linoleate, dimethicone, glycerine, and mixtures thereof. The hydrophilic agent may be hyaluronic acid, sodium peroxylinecarbolic acid, wheat protein, hair keratin amino acids, or a mixtures thereof.

The at least one additional pharmaceutical composition may be an anti-inflammatory component provided in an amount sufficient to inhibit or reduce inflammation. The at least one additional anti-inflammatory component may be aloe vera gel, aloe vera, licorice extract, pilewort, Canadian willow root, zinc, pile wort, arnica, Vitamin E, allantoin, chamomile, hydrocortisone, steroids, non-steroidal anti-inflammatory drugs, or mixtures thereof.

The at least one additional pharmaceutical composition may be an immunity boosting component in an amount sufficient to stimulate the patient's immune system response to prevent or facilitate repair of damaged skin, such as echinacea, echinacea extract, golden seal, and mixtures thereof.

The at least one additional pharmaceutical composition my be administered concurrently with the dermatological agent.

### DETAILED DESCRIPTION OF THE PREFERRED EMBODIMENTS

A dermatological agent that advantageously treats dermatological conditions has now been discovered. The dermatological agent may be used alone or in conjunction with another composition, such a sunscreen, to treat dermatological conditions. The dermatological agent includes one or more of ellagic acid, caffeic acid, tannic acid, ferulic acid, or a pharmaceutically acceptable salt thereof, and a pharmaceutically acceptable carrier. The invention also includes methods of administering a therapeutically effective amount of one or more of ellagic acid, caffeic acid, tannic acid, ferulic acid, or a pharmaceutically acceptable salt thereof, for treatment of dermatological conditions. Without wishing to be bound by theory, it is believed that the ellagic acid, caffeic acid, tannic acid, ferulic acid, or combinations thereof, advantageously treat certain dermatological conditions by strengthening cell membranes in the skin, thereby reducing, inhibiting, or avoiding the effects of the aging process. Preferably, the methods of the invention treat dermatological conditions.

Preferably, the dermatological agent of the invention is used in combination with one or more of a moisturizer, anti-inflammatory component, immunity boosting component, or an anti-oxidant.

The terms “treating” or “treatment,” as used herein, includes one or more of ameliorating or modifying a dermatological condition.

The term “dermatological conditions,” as used herein, means conditions present anywhere on the skin caused by aging or extrinsic factors such as sunlight, radiations, air pollution, wind, cold, dampness, heat, chemicals, smoke, and smoking. Dermatological conditions include, but are not limited to, dry skin; dandruff; warts; acne; keratosis, such as actinic or seborrheic keratosis; psoriasis; eczema; skin cancers; pruritus; age spots; reduced skin moisture; spider veins; senile purpura; lentigines; melasmas; deepening of skin lines; blotches; wrinkles; microbial infections; blemished skin; nodules; atrophy; rosacea; impetigo; elastotic changes characterized by leathery, course, rough, dry and yellowish skin; telangiecatic skin; hyperpigmented skin; hyperkeratotic skin; nail infections; inflammatory dermatoses; and damage to hair including, but not limited to, hair breakage, weathering damage, and thinning of hair. Preferably, the compositions and methods of the invention reduce, inhibit, or avoid wrinkles. In varying embodiments, the dermatological compositions, improve skin elasticity, improve skin softness, or both.

In a preferred embodiment of the pharmaceutical composition, the ellagic acid, caffeic acid, tannic acid, ferulic acid, or combinations thereof, is present in an amount from about 0.01 to 80 weight percent, preferably from about 0.1 to 20 weight percent, and more preferably from about 0.5 to 10 weight percent by weight of the composition. More preferably, the composition contains one of ellagic acid, tannic acid, ferullic acid, or caffeic acid. In one preferred embodiment the composition includes ellagic acid. In a second preferred embodiment the composition includes tannic acid. In a third preferred embodiment preferred embodiment the composition includes ferulic acid. In a fourth preferred embodiment the composition includes caffeic acid.

Any suitable pharmaceutically acceptable carrier may be used with the dermatological agents, as will be readily apparent to one of ordinary skill in the art. Pharmaceutically acceptable carriers include, but are not limited to, hydroxypropyl cellulose, starch (corn, potato, rice, wheat), pregelatinized starch, gelatin, sucrose, acacia, alginic acid, sodium alginate, guar gum, ethyl cellulose, carboxymethylcellulose sodium, carboxymethylcellulose calcium, polyvinylpyrrolidone, methylcellulose, hydroxyproply methylcellulose, microcrystalline cellulose, polyethylene glycol, powdered cellulose, glucose, croscarmellose sodium, crospovidone, polacrilin potassium, sodium starch glycolate, tragacanth, calcium carbonate, dibasic calcium phosphate, tribasic calcium phosphate, kaolin, mannitol, talc, cellulose acetate phthalate, polyethylene phthalate, shellac, titanium dioxide, carnauba wax, microcrystalline wax, calcium stearate, magnesium stearate, castor oil, mineral oil, light mineral oil, glycerin, sorbitol, mannitol, stearic acid, sodium lauryl sulfate, hydrogenated vegetable oil (e.g., peanut, cottonseed, sunflower, sesame, olive, corn, soybean), zinc stearate, ethyl oleate, ethyl laurate, agar, calcium silicate, magnesium silicate, silicon dioxide, colloidal silicon dioxide, calcium chloride, calcium sulfate, silica gel, castor oil, diethyl phthalate, glyercin, mono- and di-acetylated monoglycerides, propylene glycol, triacetin, alamic acid, aluminum monostearate, bentonite, bentonite magma, carbomer 934, carboxymethylcellulose sodium 12, carrageenan, hydroxyethyl cellulose, magnesium aluminum silicate, pectin, polyvinyl alcohol, povidine, sodium alginate, tragacanth, xanthan gum, and silicones. For example, preferred topical formulations of the pharmaceutical composition may include a silicon-containing carrier, preferably a silicone, but in amounts insufficient to cause substantial irritation. Suitable silicones include cyclomethicone or a mixture of cyclopentasiloxane and dimethicone/vinyldimethicone crosspolymer.

The pharmaceutical composition preferably includes one or more of a moisturizing agent, anti-inflammatory component, immunity boosting component, or additional anti-oxidant. “Moisturizing agent,” as used herein, is used to include any agent that facilitates hydration of the skin by inhibiting or preventing loss of water from the skin, absorbs water from the atmosphere and hydrates the skin, or enhances the skin's own ability to absorb water directly from the atmosphere, or a combination thereof. Moisturizing agents generally minimize or prevent the skin from drying and cracking. Cracked skin is more susceptible to environmental factors, such as those that generate free radicals, which is believed to cause further damage to the skin. Thus, moisturizing skin or facilitating moisturized skin reduces, inhibits, or avoids damage from free radicals and can help treat many dermatological conditions. Suitable moisturizing agents include, but are not limited to, acidic components, hydrophobic agents, and hydrophilic agents, or combinations thereof. Moisturizers, when used, are typically present in an amount from about 0.01 to 20 weight percent, preferably about 0.05 to 10 weight percent, more preferably from about 0.1 to 1 weight percent of the composition, although this will vary depending on the type of moisturizing agent as further noted herein..

Moisturizing agents that are acidic components include mono- or poly-hydroxy acids and mixtures thereof, or a pharmaceutically acceptable salt or ester thereof One of ordinary skill in the art will be readily able to select and prepare suitable mono- or poly-hydroxy acids for use in the composition of the invention including, for example, alkyl hydroxycarboxylic acids, aralkyl and aryl hydroxycarboxylic acids, polyhydroxy-carboxylic acids, and hydroxy-polycarboxylic acids. One of ordinary skill in the art would typically select one or more of the following mono- or poly-hydroxy acids: 2-hydroxyacetic acid (glycolic acid); 2-hydroxypropanoic acid (lactic acid); 2-methyl 2-hydroxypropanoic acid; 2-hydroxybutanoic acid; phenyl 2-hydroxyacetic acid; phenyl 2-methyl 2-hydroxyacetic acid; 3-phenyl 2-hydroxyacetic acid; 2,3-dihydroxypropanoic acid; 2,3,4-trihydroxybutanoic acid; 2,3,4,5,6-pentahydroxyhexanoic acid; 2-hydroxydodecanoic acid; 2,3,4,5-tetrahydroxypentanoic acid; 2,3,4,5,6,7-hexahydroxyheptanoic acid; diphenyl 2-hydroxyacetic acid; 4-hydroxymandelic acid; 4-chloromandelic acid; 3-hydroxybutanoic acid; 4-hydroxybutanoic acid; 2-hydroxyhexanoic acid; 5-hydroxydodecanoic acid; 12-hydroxydodecanoic acid; 10-hydroxydecanoic acid; 16-hydroxyhexadecanoic acid; 2-hydroxy-3-methylbutanoic acid; 2-hydroxy-4-methylpentanoic acid; 3-hydroxy-4-methoxymandelic acid; 4-hydroxy-3-methoxymandelic acid; 2-hydroxy-2-methylbutanoic acid; 3-(2-hydroxyphenyl) lactic acid; 3-(4-hydroxyphenyl) lactic acid; hexahydromandelic acid; 3-hydroxy-3-methylpentanoic acid; 4-hydroxydecanoic acid; 5-hydroxydecanoic acid; aleuritic acid; 2-hydroxypropanedioic acid; 2-hydroxybutanedioic acid; erythraric acid; threaric acid; arabiraric acid; ribaric acid; xylaric acid; lyxaric acid; glucaric acid; galactaric acid; mannaric acid; gularic acid; allaric acid; altraric acid; idaric acid; talaric acid; 2-hydroxy-2-methylbutanedioic acid; citric acid, isocitric acid, agaricic acid, quinic acid, glucoronic acid, glucoronolactone, galactoronic acid, galactoronolactone, uronic acids, uronolactones, ascorbic acid, dihydroascorbic acid, dihydroxytartaric acid, tropic acid, ribonolactone, gluconolactone, galactonolactone, gulonolactone, mannonolactone, citramalic acid; pyruvic acid, hydroxypyruvic acid, hydroxypyruvic acid phosphate and esters thereof, methyl pyruvate, ethyl pyruvate, propyl pyruvate, isopropyl pyruvate; phenyl pyruvic acid and esters thereof; methyl phenyl pyruvate, ethyl phenyl pyruvate, propyl phenyl pyruvate; formyl formic acid and esters thereof; methyl formyl formate, ethyl formyl formate, propyl formyl formate; benzoyl formic acid and esters thereof, methyl benzoyl formate, ethyl benzoyl formate and propyl benzoyl formate; 4-hydroxybenzoyl formic acid and esters thereof; 4-hydroxyphenyl pyruvic acid and esters thereof; and 2-hydroxyphenyl pyruvic acid and esters thereof. The hydroxy acids are preferably selected from one or more alpha-hydroxy acids or beta-hydroxy acids, more preferably from glycolic, lactic, citric, or salicylic acid, and most preferably from citric and salicylic acids. Tannic acid can also act as a moisturizer. It should be understood that one or more derivatives of the above-described acidic component, such as esters or lactones thereof, may also be suitably used. One of ordinary skill in the art will also understand that various hydroxy acids, such as those described in U.S. Pat. Nos. 5,547,988 and 5,422,370, are also suitable for use in the dermatological agents and methods of the invention.

The acidic component, when present, is typically included in the composition and methods in an amount sufficient to exfoliate, i.e., remove dead or dying skin cells, from at least a portion of the skin. By removing dead or dying skin cells, the skin is better able to absorb moisture from the atmosphere. The acidic component, when used, is typically present in an amount from about 0.1 to 12 weight percent, preferably from about 1 to 11 weight percent, more preferably from about 4 to 10 weight percent of the composition. For example, the acidic component may be present in an amount of about 0.1 to 3 weight percent citric acid in combination with up to about 2 weight percent salicylic acid.

Moisturizing agents that are hydrophobic agents include, but are not limited to, ceramide, borage oil (linoleic acid), tocopherol linoleate, dimethicone, glycerine, and mixtures thereof. Hydrophobic agents, when present, are believed to moisturize the skin by inhibiting or preventing the loss of water from the skin. The hydrophobic agent, when present, is typically present in an amount from about 0.01 to 2 weight percent, preferably from about 0.05 to 1.5 weight percent, and more preferably from about 0.1 to 1 weight percent of the composition.

Moisturizing agents that are hydrophilic agents include, but are not limited to, hyaluronic acid, sodium peroxylinecarbolic acid (sodium PCA), wheat protein (e.g., laurdimonium hydroxypropyl hydrolyzed wheat protein), hair keratin amino acids, and mixtures thereof. Sodium chloride may also be present, particularly when hair keratin amino acids are included as a moisturizer. Hydrophilic agents, when present, are believed to moisturize the skin by absorbing moisture from the atmosphere to hydrate or facilitate hydration of the skin. The hydrophilic agent, when present, is typically present in an amount from about 0.01 to 2 weight percent, preferably from about 0.05 to 1.5 weight percent, and more preferably from about 0.1 to 1 weight percent of the composition.

Other moisturizing agents that hydrate the skin and are useful in the compositions and methods of the present invention include primrose oil; GLA 3 and other fish oils that may include, for example, the omega-3 and omega-6 oils and/or linoleic acid; and flax seed oil. Preferably, these other moisturizing agents are administered orally.

In a preferred embodiment, the dermatological agent includes a mono- or poly-hydroxy acid, or a mixture thereof, or a pharmaceutically acceptable salt or ester thereof, to act as an exfoliant to facilitate removal of dead or dying skin cells and improve the skin's own ability to absorb moisture directly from the atmosphere, optionally in combination with one or more hydrophilic agents to help facilitate absorption of moisture from the atmosphere to hydrate the skin or in combination with one or more a hydrophobic agents to inhibit or prevent moisture loss by the skin.

In another embodiment, the pharmaceutical composition preferably includes one or more of an anti-inflammatory component in an amount sufficient to reduce or avoid redness and swelling of the skin, an immunity boosting component in an amount sufficient to boost the immune system to facilitate repair of damaged skin, or an antioxidant component in an amount sufficient to neutralize free radicals.

The anti-inflammatory component may prevent and reduce inflammation, including the redness and swelling that often accompanies damaged skin. Without wishing to be bound by theory, it is believed that free radical damage causes inflammation and that the one or more of ellagic acid, ferrulic acid, caffeic acid, or tannic acid facilitate neutralization of free radicals in the skin. The combination of an anti-inflammatory component with one or more of ellagic acid, ferrulic acid, caffeic acid, or tannic acid, however, is more effective at reducing, inhibiting, or avoiding inflammation than either the anti-inflammatory component or the one or more of ellagic acid, ferrulic acid, caffeic acid, or tannic acid individually.

Anti-inflammatory agents useful in the compositions and methods of the invention include any pharmaceutically acceptable compounds anti-inflammatory suitable for administration orally or topically. The anti-inflammatory component includes, but is not limited to, preferably at least one of aloe vera gel, aloe vera, licorice extract, pilewort, Canadian willow root, zinc, pile wort, arnica, allantoin, chamomile, hydrocortisone, steroids, and non-steroidal anti-inflammatory drugs (NSABD). A preferred anti-inflammatory agent is allantoin.

NSAIDs useful in the compositions and methods of the invention include, but are not limited to, salicylic acid derivatives such as aspirin, sodium salicylate, choline magnesium salicylate, salsalate, diflunisal, salicylsalicylic acid, sulfasalazine, and olsalazine; para-aminophenol derivatives such as acetaminophen; indole and indene acetic acids such as indomethacin, sulindac, and etodolac; heteroaryl acetic acids such as tolmetin, diclofenac, and ketorolac; arylpropionic acids such as ibuprofen, naproxen, flurbiprofen, ketoprofen, fenoprofen, and oxaprozin; anthranilic acids (fenamates) such as oxicams (piroxicam, tenoxicam), pyrazolidineones (phenylbutazone, oxyphenthatrazone); alkanones such as nabumetone; apazone (azapropazone); nimesulide; and combinations thereof.

The anti-inflammatory component, when present, is included in an amount sufficient to inhibit, reduce, or avoid inflammation, preferably in an amount from about 0.1 to 2 weight percent, preferably from about 0.3 to 1.5 weight percent, and more preferably from about 0.3 to 1 weight percent of the composition. When the component includes more than one anti-inflammatory compound, each compound or the total amount of the component may fall within these amounts. It should be understood, with reference to treating dermatological conditions, that the anti-inflammatory agents facilitate inhibition or suppression of inflammation anywhere on or in the skin or in adjacent bodily tissues. A transition metal component and/or vitamin E may optionally be included to assist in inhibiting or reducing inflammation, either alone or in combination with another anti-inflammatory agent.

The optional vitamin E component, when used, is preferably a sulfate or succinate vitamin E complex, or a pharmaceutically acceptable salt thereof, and more preferably a D-alpha tocopherol acid succinate. The vitamin E component, when included, is typically present in topical formulations in an amount from about 5 to 40 weight percent, preferably from about 6 to 30 weight percent, and more preferably from about 7 to 20 weight percent of the composition. When formulated in an oral preparation, the vitamin E may be present in an amount from about 1 to 60 weight percent, preferably from about 5 to 50 weight percent.

The transition metal component, preferably a zinc component, when included in the pharmaceutical composition, prevents or mitigates inflammation and assists in binding collagen fibers within the skin. Transition metals such as zinc are essential to SOD, and thus they affect the body in counteracting free radical formation. The transition metal component, when included, may be any pharmaceutically acceptable type and amount of a transition metal compound, or a pharmaceutically acceptable salt thereof. Preferably, the transition metal is complexed with an amino acid, and more preferably with monomethinone. The transition metal component, when used, is typically present in an amount from about 10 to 30 weight percent of the complex. The transition metal component, when included, is typically present in an amount from about 1 to 12 weight percent, preferably from about 1.5 to 8 weight percent, and more preferably from about 2 to 6 weight percent, of the pharmaceutical composition. A unit dose of the transition metal component is typically present in an amount from about 1 mg to 80 mg, preferably from about 2 mg to 15 mg, and more preferably for oral administration in an amount from about 5 mg to 10 mg. Although effective in helping to protect skin from damage, increasing the transition metal component concentration too much, particularly of zinc, in an oral formulation of the pharmaceutical composition may lead to stomach discomfort. One of ordinary skill in the art will be able to readily select a suitable dosage amount particularly in view of the guidelines herein.

Anti-oxidants of both the enzymatic and non-enzymatic type may be included in the dermatological agents and methods of the invention. For example, superoxide dismutase (SOD), catalase, and glutathione peroxidase are natural enzymatic anti-oxidants used by the body that may be included with the dermatological agents and pharmaceutical compositions herein. Suitable non-enzymatic anti-oxidants include Vitamin E (e.g., tocopherol), Vitamin A (retinol), Vitamin C (ascorbic acid), carotenoids, echinacoside and caffeoyl derivatives, oligomeric proanthocyanidins or proanthanols (e.g., grape seed extract), silymarin (e.g., milk thistle extract, Silybum marianum), ginkgo biloba, green tea polyphenols, and the like, and mixtures thereof. Indeed, any pharmaceutically acceptable compounds suitable for administration orally or topically may be used as an anti-oxidant in the dermatological agents of the present invention, either alone or in any combination. Preferably, the anti-oxidant component includes Vitamin E, Vitamin C, or a carotenoid. The anti-oxidant component, when used, is present in an amount sufficient to inhibit or reduce the effects of free-radicals at the skin.

The pharmaceutical composition may include a vitamin C component as an antioxidant, preferably an ascorbic acid, or a pharmaceutically acceptable salt or ester thereof, and more preferably ascorbyl palmitate, dipalmitate L-ascorbate, sodium L-ascorbate-2-sulfate, or an ascorbic salt, such as sodium, potassium, and calcium, or mixtures thereof. When oral formulations of the pharmaceutical composition are used, it is preferred that a nonacidic form of vitamin C be used to reduce the stomach irritation that may occur when using an acidic form. The vitamin C component, when used, is typically present in the pharmaceutical composition in an amount from about 0.1 to 50 weight percent, preferably from about 5 to 40 weight percent, and more preferably from about 10 to 25 weight percent.

An optional vitamin A component may also be included in the composition of the invention, and this preferably is vitamin A palmitate. The vitamin A component, when used, is typically present in an amount from about 5 to 50 weight percent, more preferably from about 6 to 40 weight percent, and most preferably from about 7 to 30 weight percent of the composition. Topical formulations of the composition, however, will typically include the vitamin A component in an amount from about 0.5 to 15 weight percent, preferably from about 1 to 10 weight percent.

Carotenoids are also antioxidants, and they include, for example, betacarotene, canthaxanthin, zeaxanthin, lycopen, lutein, crocetin, capsanthin, and mixtures thereof. Beta carotene is a carotenoid that is predominantly found in the skin. A carotenoid component, preferably beta carotene, is optionally present in an amount from about 0.1 to 5 weight percent, preferably from about 0.2 to 4 weight percent, and more preferably from about 0.3 to 3 weight percent in the pharmaceutical composition.

The pharmaceutical composition may also include a catechin-based component as an additional antioxidant. These antioxidants are believed to provide roughly 20 times more antioxidative power than vitamin C and 50 times more antioxidative power than vitamin E. The catechin-based preparation is preferably a proanthanol or a proanthocyanidin, and more preferably a proanthanol, which is commonly obtained from grape seed extract. The catechin-based preparation, when used, is typically present in an amount from about 0.1 to 5 weight percent, preferably from about 0.2 to 3 weight percent, and most preferably from about 0.3 to 2 weight percent of the composition.

The composition may also include quercetin powder as an additional antioxidant. Preferably, the quercetin powder is quercetin dihydrate. When included in the composition, the quercetin is typically present in an amount from about 1 to 20 weight percent, preferably from about 2 to 15 weight percent, and more preferably from about 3 to 10 weight percent in the pharmaceutical composition. Other forms of quercetin can be used, if desired.

A silymarin component may also be added to the pharmaceutical composition. The silymarin component provides an antioxidant component that is believed to specifically target the liver. Preferably, milk thistle extract, also known as Silybum marianum, can provide the silymarin for inclusion in the present invention. The extract itself typically contains about 70 to 95 weight percent of silymarin. The silymarin component may be present in an amount from about 0.001 to 1 weight percent, preferably from about 0.01 to 0.5 weight percent of the composition.

In another embodiment, Ginkgo Biloba extract is optionally included in the composition. Volatile oils, tannin and resin are believed to be the active constituents of the extract. Ginkgo Biloba supplies antioxidants that are believed to target the brain. Ginkgo Biloba, when used in the composition, is typically present in an amount from about 0.01 to 3 weight percent, preferably from about 0.02 to 2 weight percent, and more preferably from about 0.03 to 1 weight percent in the pharmaceutical composition.

Yet another antioxidant suitable for use in or with the composition includes a polyphenol. In one embodiment the polyphenol is provided from fruit extracts including but not limited to extracts from apricots, apples, peaches, pears, pineapples, papayas, pomegranates, cherries, kiwis, tangerines, grapes, oranges, and the like. Preferably the extract is a pomegranate extract. In another embodiment the polyphenol is provided green tea extract. Suitable additional types and amounts of anti-oxidants will be readily determinable by one of ordinary skill in the art as guided by the disclosure herein. The amount of any antioxidant component as a whole will preferably be sufficient to provide an antioxidant effect when the composition of the invention is administered.

As noted above, an immunity boosting component may also preferably be included as part of the composition. Suitable immune boosters including echinacea, golden seal, and combinations thereof, facilitate healing of the sun damaged tissues.

Echinacea and its extract are obtained from the Echinacea family of plants, and these components act as immune boosters. Also, they contain several potent antioxidant compounds, such as echinacoside and caffeoyl derivatives. Echinacea, when included, is typically present in an amount from about 1 to 20 weight percent, preferably from about 2 to 15 weight percent, and more preferably from about 3 to 10 weight percent of the composition.

An additional immunity boosting component can be provided by golden seal, also known as Hydrastis canaderis. This is optional, but preferably present in the pharmaceutical composition, typically in an amount from about 1 to 20 weight percent, preferably from about 2 to 15 weight percent, and more preferably from about 3 to 10 weight percent of the composition.

The pharmaceutical composition may further optionally include one or more of a cysteine component, magnesium component, manganese component, carotenoid component, selenium component, and copper component.

The optional cysteine component assists in thickening the dermis, supplementing of collagen and elastic tissue, and consequently, reduction of wrinkles and other skin conditions. The cysteine component, when used in the composition, is preferably N-acetyl cysteine, or a pharmaceutically acceptable salt thereof, and is then typically present in an amount from about 1 to 10 weight percent, preferably from about 2 to 8 weight percent, and more preferably from about 3 to 6 weight percent of the composition.

The optional manganese component is the co-factor used by the SOD found in mitochondria. The manganese component may be any manganese compound, or pharmaceutically acceptable salt thereof, but preferably is manganese ascorbate or a manganese ascorbic acid complex. The manganese, when present, is typically present in an amount from about 0.5 to 10 weight percent, preferably from about 1 to 8 weight percent and most preferably from about 5 to 7 weight percent, wherein the manganese is present in an amount from about 5 to 20 weight percent of a complex such as manganese ascorbate.

The copper component may also be included in the pharmaceutical composition, and may be any copper compound, or a pharmaceutically acceptable salt thereof. The copper component inhibits elastase and assists in treatment of elastic tissue defects. Preferably, the copper compound is copper sebacate. The copper, when included in the composition, is typically present in an amount from about 5 to 20 weight percent of the copper sebacate. The copper component is typically present in an amount from about 0.01 to 5 weight percent, preferably from about 0.02 to 3 weight percent, and more preferably from about 0.03 to 2 weight percent of the composition.

The magnesium component is also optional and may be any magnesium compound, or a pharmaceutically acceptable salt thereof, but preferably is magnesium ascorbate or magnesium ascorbic acid complex, wherein the magnesium is typically present in about 5 to 20 weight percent of the complex. The magnesium component, when included in the composition, is typically present in an amount from about 1 to 10 weight percent, preferably from about 3 to 8 weight percent, and more preferably from about 5 to 7 percent of the composition.

Additionally, a source of selenium may also be optionally added to the pharmaceutical composition. A selenium compound, or a pharmaceutically acceptable salt thereof, may be used. When present, the selenium compound is preferably selenium complexed with an amino acid. More preferably, the selenium compound is L-selenomethionine, wherein the selenium is present in an amount from about 0.1 to 5 weight percent of the complex. The selenium, when included, is typically present in an amount from about 0.01 to 3 weight percent, preferably from about 0.05 to 2 weight percent, and more preferably from about 0.1 to 1 weight percent in the pharmaceutical composition.

Preferably, the pharmaceutical composition also optionally includes at least one herb from the group of yellow dock, bupleurum, poria cocos, gentian root, myrr gum, hawthorn berry extract, rosemary extract, wild yam root, wild yam extract, marshmallow root, black cohosh, soy, or ginger. These pharmaceutical components are particularly useful in managing dermatological conditions resulting from exposure to ultraviolet light. The synergistic effect of these pharmaceutical components boosts the sun protection factor (SPF) of known sunscreens. Preferably, the use of one or more herbs with the composition will provide a boost of at least about 5 percent, preferably at least about 10 percent, to the SPF of a conventional sunscreen.

Yellow Dock, also known as Rumex crispus, is often used to treat skin disease, especially those involving some form of inflammation. The active constituents of yellow dock are believed to be rumicin and chrysarobin. Yellow Dock extract, when included, is typically present in the pharmaceutical composition in an amount from about 1 to 30 weight percent, preferably from about 3 to 25 weight percent, and more preferably from about 5 to 20 weight percent of the composition.

Bupleurum, also known as Bupleurum falactum, is known for its effect on the liver. The active constituents in bupleurum are believed to be furfurol, sterol, and bupleurumol. The bupleurum, when included, is typically present in the present pharmaceutical composition in an amount from about 1 to 20 weight percent weight, preferably about 2 to 15 weight percent, and more preferably from about 3 to 10 weight percent of the composition.

The active constituents in poria cocos, also known as Lycoperdon solidum, are tetracyclic titerpenic acids, polysaccharides, ergostol, choline, lipase, and protease. This herb is useful for reducing or eliminating excess fluids from the body. When included in the dermatological agents of the invention, it is typically present in an amount from about 1 to 20 weight percent, preferably from about 2 to 15 weight percent, and more preferably from about 3 to 10 weight percent of the composition.

The bitter glycosides in gentian root, also known as Gentian lutea, account for its use as a digestive bitter and liver disorder treatment. Gentian root is optionally present in the dermatological agents when used. It is typically present in an amount from about 1 to 20 weight percent, preferably from about 2 to 15 weight percent, and more preferably from about 3 to 10 weight percent of the composition.

Myrrh, also known as Commiphora myrrha, has several oils, resins and gums that increase circulation and heart rate. Myrrh gum is optionally used in the present pharmaceutical composition. When used, the gum is typically present in an amount from about 1 to 20 weight percent, preferably from about 2 to 15 weight percent, and more preferably from about 3 to 10 weight percent of the composition.

Hawthorn berry extract, also known as Crataegus supplement, can optionally be added to the pharmaceutical composition, as well. This herb is useful in the treatment of heart disease. Crategolic acid, citric acid, tartaric acid, glavone, glycosides, and vitamin C are the active constituents of hawthorne berries. The hawthorn berry extract, when included, is typically present in an amount from about 0.5 to 8 weight percent, preferably from about 0.6 to 6 weight percent, and more preferably from about 0.7 to 4 weight percent of the composition.

Rosemary contains aromatic oils that my assist with stomach disorders, and salicylic acid. When included in the composition, rosemary is typically present in an amount from about 0.5 to 8 weight percent, preferably from about 0.6 to 6 weight percent, and more preferably from about 0.7 to 4 weight percent of the composition.

Wild yam possesses glycoside saponins and diosgenins, hormonal precursors to cortical steroids that may help to reduce pain. It is believed to assist with problems of the liver and gall bladder, as well. It is optionally present in the pharmaceutical composition and, when used, is typically present in an amount from about 0.5 to 8 weight percent, preferably from about 0.6 to 6 weight percent, and more preferably from about 0.7 to 4 weight percent.

The marshmallow root, also known as Althea officinalis, acts as an antiinflammatory. The mucilage in the herb soothes membranes, thereby reducing inflammation. Marshmallow root is optionally present in the pharmaceutical composition. When included in the composition, it is typically present in an amount from about 0.5 to 8 weight percent, preferably from about 0.6 to 6 weight percent, and more preferably from about 0.7 to 4 weight percent of the composition.

Black cohosh acts as a natural estrogen supplement. Soy and ginger may act as an anti-oxidant and may act as a moisturizer to hydrate or facilitate hydration of the skin. The amount of these herbs, when present in the dermatological agents of the invention, may be readily determined by one of ordinary skill in the art.

In one embodiment the dermatological agent is used to treat a microbial infection. In the embodiment wherein the dermatological condition is a microbial infection, the dermatological agent of the invention is administered in conjunction with an antimicrobial agent. The compositions of the invention facilitate the penetration of antimicrobial agents into the skin. The dermatological agent of the invention can be administered concurrently or sequentially with the antimicrobial agent. In one embodiment the dermatological agent of the invention further comprises an antimicrobial agent. Any pharmaceutically acceptable antimicrobial agent available to those of ordinary skill in the art may be used, but preferably the antimicrobial agents used with compositions of the invention is at least one of an antibacterial agent, antifungal agent, antiviral agent, or anthelmintic. A single broad spectrum antimicrobial agent, i.e., one that is believed to have at least two of antibacterial, antifungal, and antiviral efficacy, include: echinacea, golden seal, benzalkonium chloride, benzethonium chloride, iodine, grape seed extract, pomegranate extract, green tea extract or polyphenols, and the like, or combinations thereof, may be included. Another suitable antimicrobial agent includes the class of anthelmintics, such as metronidazole, to facilitate treatment of, e.g., tricomona infection. Preferred antiviral agents include, but are not limited to, acyclovir, tamvir, penciclovir, and the like, and mixtures thereof. Preferred antibacterial agents include, but are not limited to, triclosan, neomycin, polymyxin, bacitracin, clindamycin, benzoyl peroxide, a tetracycline, a sulfa drug, a penicillin, a quinolone, a cephalosporin, and mixtures thereof. Preferred antifungal agents include, but are not limited to, farnesol, econazole, fluconazole, clotrimazole, ketoconazole, calcium or zinc undecylenate, undecylenic acid, butenafine hydrochloride, ciclopirox olaimine, miconazole nitrate, nystatin, sulconazole, terbinafine hydrochloride, and the like, and mixtures thereof. Exemplary tetracyclines include doxycycline and minocycline. An exemplary sulfa drug includes sulfacetamde. An exemplary cephalosporin includes cephalexin (commercially available as KEFLEX). Exemplary quinolones include the floxacins, such as loemfloxacin, ofloxacin, and trovafloxacin. It should be readily understood that any salts, isomers, prodrugs, metabolites, or other derivatives of these antimicrobial agents may also be included as the antimicrobial agent in accordance with the invention.

The antimicrobial agent may be included in the composition of the invention and administered with the composition of the invention or may be administered separately. When the antimicrobial agent is included in the composition of the invention it is typically present in an amount from about 0.01 to 1.5 weight percent, preferably from about 0.1 to 1.2 weight percent, and more preferably from about 0.3 to 1 weight percent of the composition. The antimicrobial agent inhibits the formation, and may further reduce, the presence of microbes that cause redness, inflammation, and irritation of the skin.

It should be understood that it is preferred that the amounts of all aspects of the composition described herein, when present, be included in an amount sufficient to perform their stated function.

The dermatological agent may also be administered concurrently or sequentially with at least one additional dermatological agent, which may include a sunscreen or sunblock, an oral or topical nutritional supplement including at least one antioxidant, or any other topical dermatological or cosmetic application. Sunscreens or sunblocks particularly suitable should include at least one of titanium dioxide, zinc oxide, talc, red veterinary petrolatum, a cinnamate, a benzone, a salicylate, a benzoic acid, or a benzophenone. An exemplary cinnamate is octyl methoxycinnamate. An exemplary benzophenone is oxybenzophenone. An exemplary benzoic acid is para-aminobenzoic acid. Exemplary salicylates include homosalicylate or octyl salicylate. Exemplary benzones include, but are not limited to oxybenzone and avobenzone (Parsol® 1789). Suitable topical applications, other than sunscreens or topical nutritional supplements, include an antioxidant and at least one of an alpha-hydroxy acid or a beta-hydroxyacid.

The term “pharmaceutically acceptable salt” refers to a salt prepared from pharmaceutically acceptable non-toxic acids or bases including inorganic or organic acids. Examples of such inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, fliroic, glutamic, benzoic, anthranilic, salicylic, phenylacetic, mandelic, embonic (pamoic), methanesulfonic, ethanesulfonic, panthenoic, benzenesulfonic, stearic, sulfanilic, algenic, and galacturonic. Examples of such inorganic bases, for potential salt formation with the sulfate or phosphate compounds of the invention, include metallic salts made from aluminum, calcium, lithium, magnesium, potassium, sodium, and zinc. Appropriate organic bases may be selected, for example from N,N-dibenzylethylenediamine, chloroprocaine, choline, diethanolamine, ethylenediamine, meglumaine (N-methylglucamine), and procaine.

The dermatological agents of the present invention are useful for treating dermatological conditions caused by aging or extrinsic factors including, but not limited to sunlight, radiations, air pollution, wind, cold, dampness, heat, chemicals, smoke, and smoking. The dermatological agents are useful in managing dry skin; dandruff; warts; acne; keratosis, such as actinic or seborrheic keratosis; psoriasis; eczema; skin cancers; pruritus; age spots; reduced skin moisture; spider veins; senile purpura; lentigines; melasmas; deepening of skin lines; blotches; wrinkles; microbial infections; blemished skin; nodules; atrophy; rosacea; impetigo; elastotic changes characterized by leathery, course, rough, dry and yellowish skin; telangiecatic skin; hyperpigmented skin; hyperkeratotic skin; nail infections; inflammatory dermatoses; damage to hair including, but not limited to, hair breakage, weathering damage, and thinning of hair; and the like. Preferred conditions for treatment include wrinkles, fine lines, poor skin softness, and poor skin elasticity.

The phrase “therapeutically effective amount” means the amount of the dermatological agent that provides a therapeutic benefit in the treatment of a dermatological condition. It should be understood by one of ordinary skill in the art that this amount will vary depending on the condition being treated and the patient and will be readily determinable by one of ordinary skill in the art.

The magnitude of a prophylactic or therapeutic dose of the dermatological agent in treating a dermatological condition will vary with the sensitivity of the person's skin and the route of administration. The dose, and perhaps the dose frequency, will also vary according to the age, body weight, and response of the individual patient. In general, the total daily dose range, for the conditions described herein, is from about 1 mg to about 20,000 mg per day administered in about one to ten doses, preferably two to eight doses. The preferred oral daily dose range should be from about 1 mg to 2,000 mg, more preferably from about 400 mg to 1,600 mg, and most preferably from about 800 mg to 1,200 mg per day. In general, a preferred topical daily dosage range, in single or divided doses, should be from about 1 mg to 20,000 mg, more preferably from about 2,000 mg to 16,000 mg, and most preferably from about 6,000 mg to 10,000 mg per day of the composition.

It is further recommended that children, patients aged over 65 years, and those with impaired renal or hepatic function initially receive low doses, and that they then be titrated based on individual response(s) or blood level(s). It may be necessary to use dosages outside these ranges in some cases, as will be apparent to those of ordinary skill in the art. Further, it is noted that the clinician or treating physician will know how and when to interrupt, adjust, or terminate therapy in conjunction with individual patient's response.

Although any suitable route of administration may be employed for providing the patient with an effective dosage of the dermatological agent according to the methods of the present invention, topical and oral administration are preferred. It should be understood that differing routes of administration may be used for the dermatological agent and the additional dermatological agent, sunscreen, sunblock, moisturizing agent, and the like. For example, the dermatological agent can be orally administered while the sunscreen is topically administered. Most moisturizing agents are preferably topically administered. Suitable routes of administration for various components of the invention include, for example, oral, rectal, parenteral, intravenous, topical, transdermal, subcutaneous, intramuscular, and similar forms of administration may also be employed. Suitable dosage forms include tablets, troches, dispersions, suspensions, solutions, aerosols, sponges, cotton applicators, capsules, patches, suppositories, and the like.

The dermatological agents used in the methods of the present invention include the active ingredients described above, and may also contain pharmaceutically acceptable carriers, excipients and the like, and optionally, other therapeutic ingredients. The dermatological agents herein may also be administered in conjunction, i.e., concurrently or sequentially, with other skin-protective pharmaceutical compositions or devices, such as a hat, umbrella, or the like.

Dermatological agents for use in the methods of the present invention suitable for oral administration include compositions, such as suspensions, solutions, elixirs, and aerosols; and may include carriers, such as starches, sugars, microcrystalline cellulose, diluents, granulating agents, lubricants, binders, disintegrating agents, and the like. In the case of oral solid preparations (such as powders, capsules, and tablets), the oral solid preparations are typically preferred over the oral liquid preparations.

Dermatological agents for use in the methods of the present invention suitable for topical administration may be presented as discrete units including aerosol sprays, each containing a predetermined amount of the active ingredient, as a powder, stick, or granules, as creams (e.g., a conditioner), pastes, gels, lotions (e.g., a sunscreen), syrups, or ointments, on sponges or cotton applicators, or as a solution or a suspension in an aqueous liquid, a non-aqueous liquid, an oil-in-water emulsion, or a water-in-oil liquid emulsion. Similarly, because of its ease of administration, a cream, lotion, or ointment represents the most advantageous topical dosage unit form.

Dermatological agents for use in the methods of the present invention suitable for oral administration may be presented as discrete units such as capsules, cachets, or tablets, or aerosol sprays, each containing a predetermined amount of the active ingredient, as a powder or granules, as creams, pastes, gels, or ointments, or as a solution or a suspension in an aqueous liquid, a non-aqueous liquid, an oil-in-water emulsion, or a water-in-oil liquid emulsion.

All such dermatological agents may be prepared by any of the methods available to the pharmaceutical art, but all methods include the step of bringing into association the carrier(s) with the active ingredient, which constitutes one or more necessary ingredients. In general, the dermatological agents are prepared by uniformly and intimately admixing the active ingredient with liquid carriers or finely divided solid carriers or both, and then, if necessary, shaping the product into the desired presentation.

For example, a tablet may be prepared by compressing or molding, optionally, with one or more accessory ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent. Desirably, each tablet, cachet or capsule contains from about 1 mg to 2,000 mg of the active ingredient.

Other suitable dosage forms include tablets, troches, capsules, patches, gel caps, magmas, lozenges, plasters, discs, suppositories, nasal or oral sprays, and the like. When an oral dosage unit form is used instead of a topical dosage form, tablets, capsules, and gel caps are preferred, in which case solid pharmaceutical carriers may be employed. If desired, tablets may be coated by standard aqueous or non-aqueous techniques.

In addition to the common dosage forms set out above, the dermatological agents for use in the methods of the present invention may also be administered by controlled release means and/or delivery devices, such as those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566 the disclosures of which are incorporated herein by express reference thereto.

The term “about,” as used herein, should be understood to refer to both numbers in a range.

What is claimed is:

1. A dermatological agent for treating a dermatological condition in a patient having skin comprising:
at least one acid comprising at least one of ferrulic acid, caffeic acid, and tannic acid and present in an amount sufficient to strengthen cell membranes in the skin;
at least one moisturizing agent present in an amount sufficient to facilitate hydration of the skin; and
a pharmaceutically acceptable carrier.

2. The dermatological agent of claim 1, wherein the at least one acid is present in an amount from about 0.01 to 80 weight percent of the dermatological agent.

3. The dermatological agent of claim 1, wherein the moisturizing agent comprises at least one of a mono- or poly-hydroxy acid, hydrophobic agent, hydrophilic agent, or mixture thereof.

4. The dermatological agent of claim 3, wherein the mono- or poly-hydroxy acid is present and comprises at east glycolic acid, lactic acid, citric acid, or salicylic acid; the hydrophobic agent is present and comprises at least one of seramide, borage oil, tocopherol linoleate, dimethicone, or glycerine; or the hydrophilic agent is present and comprises at least one of hyaluronic acid, sodium peroxylinecarbolic acid, wheat protein, or hair keratin amino acids, and mixtures therof.

5. The dermatological agent of claim 3, wherein the moisturizing agent further comprises at least one of primrose oil, GLA 3, flax seed oil, and mixtures thereof.

6. The dermatological agent of claim 1, further comprising at least one cysteine component, magnesium component, manganese component, copper component, or selenium component.

7. The dermatological agent of claim 8, wherein the optional cysteine component comprises N-acetyl cysteine and is present in an amount from about 1 to 10 weight percent; the optional magnesium component comprises magnesium ascorbate and is present in an amount from about 1 to 10 weight percent, wherein the magnesium is present in an amount from about 10 to 30 weight percent of the complex; the optional manganese component comprises manganese ascorbate and is present in an amount from about 0.5 to 10 weight percent, wherein manganese is present in an amount from about 5 to 20 weight percent of the complex; or the optional copper component comprises copper sebacate and is present in an amount from about 0.01 to 5 weight percent, wherein the copper is present in an amount from about 5 to 20 weight percent of the complex.

8. The dermatological agent of claim 1, further comprising at least one of wild yam root, wild yam extract, yellow dock, bupleurum, poria cocos, gentian root, myrrh gum, hawthorn berry extract, marshmallow root, rosemary extract, black cohosh, soy or ginger.

9. The dermatological agent of claim 8, wherein the amount of optional wild yam root, wild yam extract, marshmallow root, hawthorn berry extract, rosemary extract, or combination thereof is from about 0.5 to 8 weight percent each; the amount of optional yellow dock is from about 1 to 30 weight percent, and the amount of optional bupleurum, poria cocos, gentian root, myrrh gum, or combination thereof is from about 1 to 20 weight percent of the composition.

10. The dermatological agent of claim 1, which further comprises at least one anti-inflammatory component in an amount sufficient to reduce inflammation of the patient's skin or an antimicrobial agent.

11. The dermatological agent of claim 10, wherein the anti-inflammatory component is present in an amount from about 5 to 40 weight percent and comprises at least one of a vitamin E source, a transition metal component, aloe vera gel, aloe vera, licorice extract, pilewort, Canadian willow root, zinc, allantoin, chamomile, hydrocortisone, steroids, non-steroidal anti-inflammatory drugs, and mixtures thereof; or the antimicrobial agent comprises an antibacterial agent, an antifungal agent, an antihelmintic, or a combination thereof, in an amount sufficient to inhibit, prevent, or kill microbes.

12. The dermatological agent of claim 1, which further comprises at least one immunity boosting component in an amount sufficient to stimulate the patient's immune system response to prevent or facilitate repair of damaged skin.

13. The dermatological agent of claim 12, wherein the immunity boosting component is present in an amount from about 1 to 20 weight percent and comprises at least one immunity boosting component selected from the group of echinacea, echinacea extract, golden seal, and mixtures thereof.

14. The dermatological agent of claim 1, which further comprises at least one antioxidant.

15. The dermatological agent of claim 14, wherein the antioxidant comprises a catechin-based preparation, a vitamin A source, a ginko biloba extract, a silymarin source, a quercetin compound, a vitamin C source, or a carotenoid.

16. The dermatological agent of claim 1 adapted for oral or topical administration.

17. The dermatological agent of claim 1, wherein the at least one acid is ferrulic acid.

18. The dermatological agent of claim 1, wherein the at least one acid is caffeic acid.

19. The dermatological agent of claim 1, wherein the at least one acid is tannic acid.

20. A dermatological agent for managing a dermatological condition in a patient having skin comprising:
at least one acid selected from the group consisting of ellagic acid, ferrulic acid, caffeic acid, and tannic acid in an amount sufficient to strengthen cell membranes in the skin;
an anti-inflammatory component in an amount sufficient to inhibit or reduce inflammation; and
a pharmaceutically acceptable carrier.

21. The dermatological agent of claim 20, wherein the anti-inflammatory agent comprises one or more of aloe vera gel, aloe vera, licorice extract, pilewort, Canadian willow root, zinc, pile wort, arnica, Vitamin E, allantoin, chamomile, hydrocortisone, steroids, or non-steroidal anti-inflammatory drugs.

22. The dermatological agent of claim 20, wherein the anti-inflammatory component is present in an amount from about 0.1 to 2 weight percent of the composition

23. The dermatological agent of claim 20, further comprising one or more transition metal components or an antimicrobial agent.

24. The dermatological agent of claim 23, wherein the transition metal component comprises zinc or the antimicrobial agent comprises an antibacterial agent, an antifungal agent, an antihelmintic, or a combination thereof, in an amount sufficient to inhibit, prevent, or kill microbes.

25. The dermatological agent of claim 25, wherein the at least one acid is caffeic acid.

26. The dermatological agent of claim 20, wherein the at least one acid is tannic acid.

27. The dermatological agent of claim 20, wherein the at least one acid is ellagic acid.

28. A method for treating one or more dermatological conditions in a patient having skin which comprises administering to the patient a therapeutically effective amount of a dermatological agent comprising at least one of ellagic acid, ferrulic acid, caffeic acid, or tannic acid in an amount sufficient to strengthen cell membranes in the skin; and a pharmaceutically acceptable carrier.

29. The method of claim 28, wherein the administering is oral or topical.

30. The method of claim 28, wherein the effective amount is from about 1 mg to 2,000 mg per day.

31. The method of claim 28, further comprising administering at least one additional pharmaceutical composition to facilitate treatment of the dermatological condition.

32. The method of claim 31, wherein the at least one additional pharmaceutical composition is a moisturizing agent provided in an amount sufficient to facilitate hydration of the skin.

33. The method of claim 31, wherein the moisturizing agent comprises a mono- or poly-hydroxy acid, a hydrophobic agent, hydrophilic agent, primrose oil, GLA 3, flax seed oil, and mixtures thereof

34. The method of claim 33, wherein the mono- or poly-hydroxy acid is selected from the group consisting of glycolic acid, lactic acid, citric acid, salicylic acid, and mixtures thereof, the hydrophobic agent is selected from the group consisting of ceramide, borage oil, tocopherol linoleate, dimethicone, glycerine, and mixtures thereof; and the hydrophilic agent is selected from the group consisting hyaluronic acid, sodium peroxylinecarbolic acid, wheat protein, hair keratin amino acids, and mixtures thereof.

35. The method of claim 31, wherein the at least one additional pharmaceutical composition is an anti-inflammatory component provided in an amount sufficient to inhibit or reduce inflammation.

36. The method of claim 35, wherein the at least one additional anti-inflammatory component is selected from the group consisting of aloe vera gel, aloe vera, icorice extract, pilewort, Canadian willow root, zinc, pile wort, arnica, Vitamin E, allantoin, hamomile, hydrocortisone, steroids, non-steroidal anti-inflammatory drugs, and mixtures hereof.

37. The method of claim 36, wherein the acid comprises ellagic acid.

38. The method of claim 36, wherein the acid comprises ferrulic acid.

39. The method of claim 36, wherein the acid comprises caffeic acid.

40. The method of claim 36, wherein the acid comprises tannic acid.

41. The method of claim 31, wherein the at least one additional pharmaceutical composition is an immunity boosting component in an amount sufficient to stimulate the patient's immune system response to prevent or facilitate repair of damaged skin.

42. The method of claim 41, wherein the immunity boosting component is selected from the group consisting of echinacea, echinacea extract, golden seal, and mixtures thereof.

43. The method of claim 31, wherein the a t least one additional pharmaceutical composition is administered concurrently with the dermatological agent.
